(19) United States
(12) Patent Application Publication
(10) Pub. No.: US 2018/0147183 A1
(43) Pub. Date: May 31, 2018

(54) USE OF R-OXIRACETAM IN PHARMACEUTICAL FIELD

(71) Applicant: Chongqing Runze Pharmaceutical Co., Ltd., Chongqing (CN)

(72) Inventor: Lei Ye, Chongqing (CN)

(21) Appl. No.: 15/574,846

(22) PCT Filed: May 17, 2016
(86) PCT No.: PCT/CN2016/082396

§ 371 Date: Nov. 17, 2017

(30) Foreign Application Priority Data
May 18, 2015 (CN) 2015102535566

(51) Int. Cl. A61K 31/4015; A61P 25/08

(52) U.S. Cl. CPC A61K 31/4015; A61P 25/08

(57) ABSTRACT

Provided is a use of R-oxiracetam in the preparation of a drug for preventing or treating epilepsy. An experimental result shows that the R-oxiracetam has an obvious effect in the treatment of generalized epilepsy seizure, partial epilepsy seizure and status epilepticus.

### TECHNICAL FIELD

The present invention relates to a use of R-oxiracetam in pharmaceutical field.

### DESCRIPTION OF RELATED ARTS

R-oxiracetam ((R)-4-hydroxy-2-oxo-1-pyrrolidineacetamide) is the R-isomer of oxiracetam (Oxiracetam CAS 62613-82-5), CN102603607A discloses the preparation method. Compared with the racemic oxiracetam and S-oxiracetam, both of the racemic oxiracetam and S-oxiracetam can enhance memory, improve learning ability, improve the cognitive function of patients, and R-oxiracetam is not effective in cognitive dysfunction. There is no report on the use of R-oxiracetam alone as medicament application.

It is known in the industry that epilepsy is common in repeated attacks with loss of consciousness as well as loss of convulsions as the main symptoms of chronic neurological diseases, and its seizures are usually caused by a short-term abnormal synchronization activity of neurons in the brain; nevertheless its specific pathogenesis is still unclear. As a common disease, according to the World Health Organization statistics, about 50 million people around the world suffer from epilepsy. Epilepsy can occur at any time no warning before the general attack thereby resulting in great harm. Furthermore, epilepsy is often accompanied by depression, anxiety, migraine, infertility, low libido, autism and other complications, and therefore, treatment of epilepsy is very difficult.

At present, for antiepileptic drugs are phenytoin sodium, sodium valproate, carbamazepine, phenobarbital, lamotrigine, levetiracetam and so on. Clinical reactions show that these drugs have many side effects, mainly for mental development, language expression, learning and memory and other cognitive decline and craniofacial deformities, congenital heart defects, toe development and other fetal malformations. It has been reported that oxiracetam is used in the treatment of epilepsy patients for the treatment of cognitive and behavioral disorders after epilepsy. In essence, antiepileptic drugs play an inhibitory role in the central nervous system. Racemic oxiracetam enhance nerve excitement conduction, promote nervous system activity, enhance memory, improve learning ability, and improve forgetfulness. Thus, oxiracetam does not have antiepileptic effect. The development of new antiepileptic drugs is necessary.

### SUMMARY OF THE INVENTION

The object of the present invention is to provide use of R-oxiracetam in the pharmaceutical field, specifically to provide a use of R-oxiracetam compound in preparation of a drug for preventing or treating epilepsy.

The present invention is specifically related to a use of R-oxiracetam in the preparation of a drug for preventing or treating epilepsy, in the preparation of a drug for preventing or treating acute epilepsy seizure.

The present invention is specifically related to a use of R-oxiracetam compound in the preparation of a drug for preventing or treating epilepsy, in the preparation of a drug for preventing or treating generalized epilepsy seizure.

The above-mentioned uses of the R-oxiracetam compound in the preparation of a drug for preventing or treating epilepsy, specifically is the preparation of a composition having R-oxiracetam compound as an active ingredient. The dosage form of the drug may be an oral preparation such as a tablet, a dripping pill, a powder, a granule, a capsule, and the like; injections such as a powder for injection and a lyophilized powder for injection. The above dosage forms may be prepared according to a conventional method.

The above dosage forms are preferably oral capsules, tablets and injections.

The dosage of the above-mentioned R-oxiracetam composition is 400 mg or more (including 400 mg), preferably 400 to 2000 mg/day, and more preferably 800 to 1600 mg/day.

The present invention is particularly to a use of R-oxiracetam compound or a composition containing R-oxiracetam compound in the preparation of a drug for preventing or treating epilepsy.

A composition for preventing or treating epilepsy contains R-oxiracetam compound and a pharmaceutically acceptable excipient.

A method for preparing a composition for preventing or treating epilepsy includes preparing the composition using the R-oxiracetam compound and a pharmaceutically acceptable excipient.

The present invention is related to a use of R-oxiracetam compound or a composition containing R-oxiracetam compound in the preparation of a drug for preventing or treating acute epilepsy seizure.

A composition for preventing or treating acute epilepsy seizure contains R-oxiracetam compound and a pharmaceutically acceptable excipient.

A method for preparing a composition for preventing or treating acute epilepsy seizure includes preparing the composition using the R-oxiracetam compound and a pharmaceutically acceptable excipient.

The present invention is related to a use of R-oxiracetam compound or a composition containing R-oxiracetam compound in the preparation of a drug for preventing or treating generalized epilepsy seizure.

A composition for preventing or treating generalized epilepsy seizure contains R-oxiracetam compound and a pharmaceutically acceptable excipient.

A method for preparing a composition for preventing or treating generalized epilepsy seizure includes preparing the composition using the R-oxiracetam compound and a pharmaceutically acceptable excipient.

The present invention is related to a use of R-oxiracetam compound or a composition containing R-oxiracetam compound in the preparation of a drug for preventing or treating partial epilepsy seizure.

A composition for preventing or treating partial epilepsy seizure contains R-oxiracetam compound and a pharmaceutically acceptable excipient.

A method for preparing a composition for preventing or treating partial epilepsy seizure includes preparing the composition using the R-oxiracetam compound and a pharmaceutically acceptable excipient.

The present invention is related to a use of R-oxiracetam compound or a composition containing R-oxiracetam compound in the preparation of a drug for preventing or treating status epilepticus.

A composition for preventing or treating status epilepticus contains R-oxiracetam compound and a pharmaceutically acceptable excipient.

A method for preparing a composition for preventing or treating status epilepticus includes preparing the composition using the R-oxiracetam compound and a pharmaceutically acceptable excipient.

A method of treating epilepsy comprising administering R-oxiracetam to a patient; the mode of administration is preferably oral or injection.

According to the preparation of the composition containing the R-oxiracetam compound, the optical purity of the R-oxiracetam compound is 99.0% or more, based on weight percent.

Stages: epileptic seizures evaluation are categorized by Racine Stages

Stage 0: no signs of any seizure;

Stage I: staring, head and face slightly trembling;

Stage II: head nodding or wet dog-like shaking;

Stage III: forelimb limit convulsions;

Stage IV: full-body ankylosing convulsions with a standing hind limb;

Stage V: full-body ankylosing clonic seizures with standing with fell;

Partial epilepsy seizure occurs when any one of Stage Ito Stage III appears;

Generalized epilepsy seizure occurs when Stage IV or Stage V appears;

Status epilepticus (SE) occurs when Stage IV or Stage V lasts 30 minutes.

To further verifying the pharmaceutical effect of the present invention, the inventors have conducted the following tests.

A. The Pharmacokinetics of R-Oxiracetam In Vivo and Studies of Bioavailability

1. Materials

1.1 Drugs

1.1.1 Standards:

Oxiracetam and R-oxiracetam are purchased from Chongqing Dongze Pharmaceutical Science and Technology Co., Ltd., lot number: oxiracetam: 20080917; R-oxiracetam: 20100205.

1.1.2 Internal Standard:

Internal Standard: Piracetam, lot number 100386-200702 (content 100.0%), purchased from NIFDC.

3.2 Reagent

Methanol (MERCK, for liquid chromatography), water (MILLI Q), acetonitrile (TEDIA, for liquid chromatography).

3.3 Instrument

SHIMADZU LC-MS single quadrupole mass spectrometer, Eppendorf 5430 desktop high speed centrifuge, XW-80A vortex mixer, SE812 nitrogen blowing instrument and SE812J numerical control constant temperature water bath (Beijing Ferren Science & Technology Co. Ltd.).

4 Determination Methods of Oxiracetam and R-Oxiracetam in Plasma by LC-MS

4.1 Chromatographic Conditions

Mobile phase: methanol: water (5:95)

Column: ZORBAX SB-Aq (2.1×100 mm 3.5μ)

Flow rate: 0.1 mL/min;

Column temperature: 30° C.;

Injection volume: 10 L 4.2 Mass Spectrometry Detection Parameters

Mass spectrometry ionization method: electrospray ionization (ESI); ion polarity Positive; scanning mode for multiple reactions monitoring (MRM); scanning time of 100 ms. The ionic reactions used for quantitative analysis were m/z (oxiracetam) m/z 159.0→m/z 113.9 and m/z 143.1→m/z 126.0 (piracetam), respectively.

4.3 Preparation of Standard Solutions

Preparation of oxiracetam standard: oxiracetam standard of 100.01 mg is accurately weighed by weight loss method, dissolved with ultrapure water, diluted in 100.0 mL volumetric flask, and formulated into 1.0001 mg/mL stock solution, and diluted to the appropriate concentration with ultra-pure water upon use.

Preparation of R-oxiracetam standard: R-oxiracetam standard of 100.01 mg is accurately weighed by weight loss method, dissolved with ultrapure water, diluted in 100.0 mL volumetric flask, and formulated into 1.0002 mg/mL stock solution, and diluted to the appropriate concentration with ultra-pure water upon use.

Internal standard solution: piracetam of 20.02 mg is accurately weighed by weight loss method, dissolved with ultrapure water, diluted in 100.0 mL volumetric flask, and formulated into 200.2 mg/mL stock solution, and diluted to the appropriate concentration with ultra-pure water upon use

4.4 Extraction of Plasma Samples

20 μL of the drug-containing the plasma sample is taken in a 1.5 mL Eppendorf tube. 20 μL of piracetam (20 μg/mL) of the internal standard solution is added, and mixed by the vortex mixer for 30s. Then, 500 μL of acetonitrile is added thereto, vortex shaken for 5 min and centrifugated at 16000 rpm for 10 min. The supernatant from the centrifugation was passed through 1 ml of SPE C 18E column and the filtrate was collected, put into 38° C. constant temperature water bath and dried by nitrogen gas. 300 μL of mobile phase was dissolved and the injection volume was 10 μL.

5 Results of Toxicokinetic Samples

5.1 Oxiracetam

5.1.1 Preparation of Standard Solutions and its Results

20 μL of blank dog plasma was added into the working solution prepared from the oxiracetam standard. The concentrations of oxiracetam in the plasma were 0.167, 0.33, 0.37, 1.33, 2.67, 4.0, 6.67, 13.33 and 26.67 μg/mL, respectively. A sample was made for each concentration respectively according to the procedures“4.4 Extraction of plasma samples”. The ratio of the peak area (As) of oxiracetam to the peak area (Ai) of the internal standard piracetam (f=As/Ai) were calculated, and perform linear regression using the concentration (C) to the ratio (f). The weight parameter 1/x, and the oxiracetam regression equation: y=5.2117X−0.76985 (r=0.9970, weight 1/x).

TABLE 1
Data sheet of standard curve of oxiracetam

| C(μg/ml) | As | Ai | f |
|---|---|---|---|
| 0.167 | 992136 | 16622206 | 0.059687 |
| 0.333 | 1549767 | 16609126 | 0.093308 |
| 0.667 | 3017344 | 10076722 | 0.299437 |
| 1.333 | 5274759 | 11128797 | 0.473974 |
| 2.667 | 9062878 | 12423386 | 0.729501 |
| 4 | 12074304 | 10813359 | 1.11661 |
| 6.667 | 17499548 | 11720309 | 1.493096 |
| 13.33 | 28478007 | 11472202 | 2.482349 |
| 26.67 | 59380130 | 11215251 | 5.294588 |

The diagram of standard curve of oxiracetam is shown as FIG. 1.

5.1.2 The Results from the Assays of Oxiracetam Sample

TABLE 2
Data sheet of the blood assay of Dog 1-2

| | | | | | Concentration |
|---|---|---|---|---|---|
| | | | | | in dog |
| | | | | | blood C |
| Time(h) | As | Ai | f | C(μg/ml) | [symbol] (μg/ml) |
| 0 | 0 | 1273566 | 0 | 0 | 0 |
| 0.25 | 276464 | 1315155 | 0.210214 | 0.325722 | 4.885835 |
| 0.5 | 732721 | 1441293 | 0.508378 | 1.879661 | 28.19492 |
| 0.75 | 963865 | 1016862 | 0.947882 | 4.170226 | 62.55338 |
| 1 | 1995011 | 1321985 | 1.509103 | 7.09514 | 106.4271 |
| 1.25 | 1762997 | 1190761 | 1.480563 | 6.946402 | 104.196 |
| 1.5 | 2614193 | 1277486 | 2.046357 | 9.895151 | 148.4273 |
| 2 | 2488246 | 1126617 | 2.2086 | 10.74071 | 161.1106 |
| 4 | 946639 | 1157519 | 0.817817 | 3.492368 | 52.38552 |
| 6 | 370351 | 1451722 | 0.255112 | 0.559715 | 8.39572 |
| 8 | 257446 | 1372566 | 0.187565 | 0.207685 | 3.115275 |
| 12 | 67028 | 768664 | 0.087201 | −0.31539 | −4.7308 |
| 24 | 0 | 987967 | 0 | 0 | 0 |

TABLE 3
Data sheet of the blood assay of Dog 1-1

| | | | | | Concentration |
|---|---|---|---|---|---|
| | | | | | in dog |
| | | | | | blood C |
| Time(h) | As | Ai | f | C(μg/ml) | [symbol] (μg/ml) |
| 0 | 0 | 886512 | 0 | 0 | 0 |
| 0.25 | 268473 | 1258959 | 0.21325 | 0.341545 | 5.123175 |
| 0.5 | 902927 | 1326947 | 0.680454 | 2.776474 | 41.64712 |
| 0.75 | 1070002 | 1390229 | 0.769659 | 3.241381 | 48.62071 |
| 1 | 1732865 | 1199276 | 1.444926 | 6.760671 | 101.4101 |
| 1.25 | 1465094 | 1282962 | 1.141962 | 5.181714 | 77.72571 |
| 1.5 | 1378168 | 804776 | 1.712486 | 8.155116 | 122.3267 |
| 2 | 2314341 | 1147313 | 2.017184 | 9.743106 | 146.1466 |
| 4 | 883382 | 1325842 | 0.66628 | 2.702601 | 40.53902 |
| 6 | 252446 | 1240015 | 0.203583 | 0.291164 | 4.367454 |
| 8 | 135200 | 1380872 | 0.097909 | −0.25958 | −3.89365 |
| 12 | 111511 | 1209146 | 0.092223 | −0.28921 | −4.33818 |
| 24 | 0 | 644736 | 0 | 0 | 0 |

TABLE 4
Data sheet of the blood assay of Dog 2-6

| | | | | | Concentration |
|---|---|---|---|---|---|
| | | | | | in dog |
| | | | | | blood C |
| Time(h) | As | Ai | f | C(μg/ml) | [symbol] (μg/ml) |
| 0 | 0 | 1168679 | 0 | 0 | 0 |
| 0.25 | 88019 | 1042543 | 0.084427 | −0.32984 | −4.94761 |
| 0.5 | 711568 | 1159296 | 0.613793 | 2.429056 | 36.43584 |
| 0.75 | 509831 | 343805 | 1.482907 | 6.958619 | 104.3793 |
| 1 | 1765579 | 1518281 | 1.16288 | 5.290733 | 79.361 |
| 1.25 | 2099611 | 1382506 | 1.518699 | 7.145156 | 107.1773 |
| 1.5 | 1807432 | 1213844 | 1.489015 | 6.99045 | 104.8567 |
| 2 | 896293 | 624506 | 1.435203 | 6.709998 | 100.65 |
| 4 | 677360 | 1308017 | 0.517853 | 1.929042 | 28.93564 |
| 6 | 106646 | 505852 | 0.210825 | 0.328904 | 4.933561 |
| 8 | 87006 | 940861 | 0.092475 | −0.2879 | −4.31848 |
| 12 | 44146 | 1048738 | 0.042094 | −0.55047 | −8.257 |
| 24 | 0 | 644736 | 0 | 0 | 0 |

TABLE 5
Data sheet of the blood assay of Dog 2-5

| | | | | | Concentration |
|---|---|---|---|---|---|
| | | | | | in dog |
| | | | | | blood C |
| Time(h) | As | Ai | f | C(μg/ml) | [symbol] (μg/ml) |
| 0 | 0 | 1341040 | 0 | 0 | 0 |
| 0.25 | 266991 | 1093498 | 0.244162 | 0.502651 | 7.539761 |
| 0.5 | 664555 | 969705 | 0.685317 | 2.801815 | 42.02722 |
| 0.75 | 839446 | 1203805 | 0.697327 | 2.86441 | 42.96615 |
| 1 | 576413 | 639882 | 0.900811 | 3.924909 | 58.87363 |
| 1.25 | 1346311 | 1315636 | 1.023316 | 4.563365 | 68.45047 |
| 1.5 | 1685657 | 1215516 | 1.386783 | 6.457647 | 96.86471 |
| 2 | 1344007 | 479583 | 2.802449 | 13.83567 | 207.5351 |
| 4 | 543839 | 783969 | 0.6937 | 2.845504 | 42.68256 |
| 6 | 315813 | 1279837 | 0.24676 | 0.516191 | 7.742862 |
| 8 | 112798 | 1303094 | 0.086562 | −0.31872 | −4.78075 |
| 12 | 130427 | 1187193 | 0.109862 | −0.19728 | −2.95926 |
| 24 | 0 | 1156095 | 0 | 0 | 0 |

TABLE 6
Data sheet of the blood assay of Dog 3-3

| | | | | | Concentration |
|---|---|---|---|---|---|
| | | | | | in dog blood |
| Time(h) | As | Ai | f | C(μg/ml) | C [symbol] (μg/ml) |
| 0 | 0 | 1423765 | 0 | 0 | 0 |
| 0.25 | 65535 | 691180 | 0.094816 | −0.2757 | −4.13545 |
| 0.5 | 874164 | 1282444 | 0.681639 | 2.782649 | 41.73973 |
| 0.75 | 1510853 | 1217024 | 1.241432 | 5.700123 | 85.50185 |
| 1 | 1834880 | 946744 | 1.938095 | 9.330921 | 139.9638 |
| 1.25 | 5184125 | 1291411 | 4.014311 | 20.15153 | 302.273 |
| 1.5 | 2450710 | 939550 | 2.608387 | 12.82428 | 192.3642 |
| 2 | 1717067 | 901121 | 1.905479 | 9.160934 | 137.414 |
| 4 | 544114 | 1304058 | 0.417247 | 1.404715 | 21.07073 |
| 6 | 746143 | 1412032 | 0.528418 | 1.984106 | 29.76158 |
| 8 | 186413 | 1349073 | 0.138179 | −0.0497 | −0.74557 |
| 12 | 91699 | 1373710 | 0.066753 | −0.42195 | −6.32932 |
| 24 | 0 | 1432647 | 0 | 0 | 0 |

TABLE 7
Data sheet of the blood assay of Dog 3-4

| | | | | | Concentration |
|---|---|---|---|---|---|
| | | | | | in dog blood |
| Time(h) | As | Ai | f | C(μg/ml) | C [symbol] (μg/ml) |
| 0 | 0 | 271384 | 0 | 0 | 0 |
| 0.25 | 73940 | 347373 | 0.212855 | 0.339485 | 5.092278 |
| 0.5 | 316630 | 459251 | 0.689449 | 2.82335 | 42.35025 |
| 0.75 | 717070 | 637701 | 1.124461 | 5.090504 | 76.35756 |
| 1 | 3905447 | 1012257 | 3.858158 | 19.33771 | 290.0656 |
| 1.25 | 1355434 | 688449 | 1.968823 | 9.491063 | 142.3659 |
| 1.5 | 877979 | 346678 | 2.532549 | 12.42904 | 186.4355 |
| 2 | 1747729 | 570226 | 3.064976 | 15.20389 | 228.0583 |
| 4 | 145610 | 255476 | 0.569956 | 2.200588 | 33.00882 |
| 6 | 121522 | 478627 | 0.253897 | 0.553385 | 8.300782 |
| 8 | 84930 | 599118 | 0.141758 | −0.03105 | −0.46572 |
| 12 | 104072 | 768305 | 0.135457 | −0.06389 | −0.95836 |
| 24 | 269524 | 634460 | 0.424808 | 1.444124 | 21.66187 |

5.2 R-Oxiracetam

5.2.1 Preparation of Standard Curves and its Results

20 μL of blank dog plasma was added into the working solution prepared from the R-oxiracetam standard. The concentrations of R-oxiracetam in the plasma were 0.167, 0.33, 0.37, 1.33, 2.67, 4.0, 6.67, 13.33 and 26.67 μg/mL, respectively. A sample was made for each concentration respectively according to the procedures “4.4 Extraction of plasma samples”. The ratio of the peak area (As) of R-oxiracetam to the peak area (Ai) of the internal standard piracetam (f=As/Ai) were calculated, and perform linear regression using the concentration (C) to the ratio (f). The weight parameter 1/x, and the oxiracetam regression equation: y=1.6811X−0.031244(r=0.9949, weight 1/x).

TABLE 8
Data sheet of standard curve of R-oxiracetam

| C(μg/ml) | As | Ai | f |
|---|---|---|---|
| 0.067 | 812704 | 16331255 | 0.049764 |
| 0.167 | 1824692 | 17170981 | 0.106266 |
| 0.33 | 3048924 | 17352720 | 0.175703 |
| 0.67 | 3001823 | 11162172 | 0.268928 |
| 1.33 | 5321083 | 7730720 | 0.688304 |
| 2.67 | 8933205 | 6252168 | 1.428817 |
| 4.0 | 12411781 | 4181600 | 2.968189 |
| 6.67 | 17957473 | 4249716 | 4.22557 |
| 13.33 | 29669086 | 3881626 | 7.643468 |

The diagram of standard curve of R-oxiracetam is shown as FIG. 2.

5.2.2 The Results from the Assays of Oxiracetam Sample

TABLE 9
Data sheet of the blood assay of Dog 1-5

| | | | | | Concentration |
|---|---|---|---|---|---|
| | | | | | in dog blood |
| Time(h) | As | Ai | f | C(μg/ml) | C [symbol] (μg/ml) |
| 0 | 0 | 551666 | 0 | 0 | 0 |
| 0.25 | 480732 | 1255106 | 0.383021 | 0.612653 | 9.18979 |
| 0.5 | 1166819 | 952801 | 1.22462 | 2.027464 | 30.41197 |
| 0.75 | 2460020 | 1277065 | 1.926308 | 3.207072 | 48.10608 |
| 1 | 2590580 | 1272425 | 2.035939 | 3.391373 | 50.8706 |
| 1.25 | 1853588 | 485827 | 3.815325 | 6.382699 | 95.74049 |
| 1.5 | 3877552 | 883340 | 4.389648 | 7.348194 | 110.2229 |
| 2 | 1009044 | 297782 | 3.388533 | 5.665218 | 84.97827 |
| 4 | 866497 | 1160418 | 0.746711 | 1.224052 | 18.36078 |
| 6 | 274483 | 853889 | 0.32145 | 0.509146 | 7.637195 |
| 8 | 107211 | 651065 | 0.16467 | 0.245583 | 3.683746 |
| 12 | 72049 | 873862 | 0.082449 | 0.107361 | 1.610414 |
| 24 | 0 | 1161598 | 0 | 0 | 0 |

TABLE 10
Data sheet of the blood assay of Dog 1-6

| | | | | | Concentration |
|---|---|---|---|---|---|
| | | | | | in dog blood |
| Time(h) | As | Ai | f | C(μg/ml) | C [symbol] (μg/ml) |
| 0 | 0 | 672215 | 0 | 0 | 0 |
| 0.25 | 60261 | 488100 | 0.12346 | 0.176305 | 2.644578 |
| 0.5 | 359624 | 956443 | 0.376001 | 0.600852 | 9.012782 |
| 0.75 | 844158 | 1176092 | 0.717765 | 1.175391 | 17.63087 |
| 1 | 1618357 | 1132934 | 1.428465 | 2.370149 | 35.55224 |
| 1.25 | 1855341 | 1265601 | 1.465976 | 2.433209 | 36.49813 |
| 1.5 | 2010997 | 1322332 | 1.520796 | 2.525366 | 37.88049 |
| 2 | 3237253 | 1200625 | 2.696307 | 4.501517 | 67.52275 |
| 4 | 835318 | 901936 | 0.926139 | 1.525688 | 22.88532 |
| 6 | 170377 | 295516 | 0.576541 | 0.937979 | 14.06968 |
| 8 | 131752 | 638978 | 0.206192 | 0.315385 | 4.730775 |
| 12 | 86278 | 913399 | 0.094458 | 0.12755 | 1.913244 |
| 24 | 0 | 1045161 | 0 | 0 | 0 |

TABLE 11
Data sheet of the blood assay of Dog 2-3

| | | | | | Concentration |
|---|---|---|---|---|---|
| | | | | | in dog blood |
| Time(h) | As | Ai | f | C(μg/ml) | C [symbol] (μg/ml) |
| 0 | 0 | 751840 | 0 | 0 | 0 |
| 0.25 | 702738 | 1144185 | 0.614182 | 1.001258 | 15.01886 |
| 0.5 | 2005286 | 981191 | 2.043726 | 3.404465 | 51.06697 |
| 0.75 | 2432070 | 813496 | 2.989652 | 4.99466 | 74.9199 |
| 1 | 3448056 | 1222672 | 2.820099 | 4.709624 | 70.64436 |
| 1.25 | 3208841 | 1124874 | 2.852623 | 4.7643 | 71.4645 |
| 1.5 | 3021390 | 992719 | 3.04355 | 5.085268 | 76.27902 |
| 2 | 3072138 | 1172934 | 2.619191 | 4.371878 | 65.57817 |
| 4 | 750345 | 1001486 | 0.749232 | 1.228289 | 18.42434 |
| 6 | 236168 | 469767 | 0.502734 | 0.813903 | 12.20854 |
| 8 | 116823 | 309924 | 0.376941 | 0.602431 | 9.036468 |
| 12 | 92460 | 871874 | 0.106047 | 0.147032 | 2.205485 |
| 24 | 0 | 465528 | 0 | 0 | 0 |

TABLE 12
Data sheet of the blood assay of Dog 2-4

| | | | | | Concentration |
|---|---|---|---|---|---|
| | | | | | in dog blood |
| Time(h) | As | Ai | f | C(μg/ml) | C [symbol] (μg/ml) |
| 0 | 0 | 1221788 | 0 | 0 | 0 |
| 0.25 | 114587 | 1174751 | 0.097542 | 0.132733 | 1.990996 |
| 0.5 | 309169 | 961759 | 0.321462 | 0.509166 | 7.637487 |
| 0.75 | 769661 | 750649 | 1.025327 | 1.692434 | 25.38651 |
| 1 | 646555 | 335063 | 1.929652 | 3.212694 | 48.19041 |
| 1.25 | 1517117 | 774517 | 1.958791 | 3.26168 | 48.9252 |
| 1.5 | 1556348 | 523101 | 2.975234 | 4.970422 | 74.55633 |
| 2 | 1796540 | 589667 | 3.046703 | 5.090568 | 76.35852 |
| 4 | 605421 | 743917 | 0.813829 | 1.336883 | 20.05325 |
| 6 | 219353 | 1081137 | 0.202891 | 0.309836 | 4.647542 |
| 8 | 130321 | 1179320 | 0.110505 | 0.154526 | 2.317895 |
| 12 | 68710 | 897659 | 0.076544 | 0.097433 | 1.4615 |
| 24 | 0 | 1202856 | 0 | 0 | 0 |

TABLE 13
Data sheet of the blood assay of Dog 3-1

| | | | | | Concentration |
|---|---|---|---|---|---|
| | | | | | in dog blood |
| Time(h) | As | Ai | f | C(μg/ml) | C [symbol] (μg/ml) |
| 0 | 0 | 58780 | 0 | 0 | 0 |
| 0.25 | 278520 | 782480 | 0.355945 | 0.567135 | 8.507032 |
| 0.5 | 257902 | 194291 | 1.327401 | 2.200249 | 33.00374 |
| 0.75 | 668626 | 333408 | 2.005429 | 3.340082 | 50.10123 |
| 1 | 2113431 | 965118 | 2.189816 | 3.650056 | 54.75084 |
| 1.25 | 2462010 | 1055741 | 2.332021 | 3.889116 | 58.33674 |
| 1.5 | 2457765 | 993482 | 2.47389 | 4.127612 | 61.91418 |
| 2 | 1235252 | 428685 | 2.881491 | 4.812831 | 72.19246 |
| 4 | 1231620 | 1949894 | 0.631634 | 1.030596 | 15.45895 |
| 6 | 259097 | 616016 | 0.420601 | 0.675828 | 10.13743 |
| 8 | 233363 | 1082583 | 0.215561 | 0.331136 | 4.967042 |
| 12 | 103250 | 644955 | 0.160089 | 0.237881 | 3.568216 |
| 24 | 0 | 1202856 | 0 | 0 | 0 |

TABLE 14
Data sheet of the blood assay of Dog 3-2

| | | | | | Concentration |
|---|---|---|---|---|---|
| | | | | | in dog blood |
| Time(h) | As | Ai | f | C(μg/ml) | C [symbol] (μg/ml) |
| 0 | 0 | 508668 | 0 | 0 | 0 |
| 0.25 | 1172419 | 478962 | 2.447833 | 4.083808 | 61.25712 |
| 0.5 | 746835 | 250642 | 2.979688 | 4.97791 | 74.66865 |
| 0.75 | 2335279 | 1024693 | 2.279004 | 3.799989 | 56.99983 |
| 1 | 1264281 | 283050 | 4.466635 | 7.477616 | 112.1642 |
| 1.25 | 975731 | 349361 | 2.792902 | 4.663903 | 69.95855 |
| 1.5 | 1414091 | 604412 | 2.339614 | 3.901882 | 58.52823 |
| 2 | 874924 | 572365 | 1.528612 | 2.538506 | 38.07758 |
| 4 | 616581 | 1189738 | 0.518249 | 0.839985 | 12.59978 |
| 6 | 226864 | 1172950 | 0.193413 | 0.293903 | 4.408544 |
| 8 | 129261 | 1214800 | 0.106405 | 0.147634 | 2.214506 |
| 12 | 81538 | 1141148 | 0.071453 | 0.088875 | 1.333125 |
| 24 | 0 | 588337 | 0 | 0 | 0 |

The major pharmacokinetic parameters of oxiracetam and R-oxiracetam in dogs are as follow: Tmax were (0.022±0.131), (2.039±0.401), (16.37±0.408) h, respectively, and the Cmax were (202.383±79.525), (85.790±19.953) mg/ml, T1/h; AUCO-∞ were (445.340±86.609), (242.262±31.343) mg*h/ml, respectively. The relative bioavailability of R-oxiracetam was (112.2±11.6) % with oxiracetam as a reference. There was no significant difference between the nonparametric test results (P>0.05).

B. Toxicological Test

The mouse toxicity of R-oxiracetam and oxiracetam was compared under GLP assay conditions. The dose is set to 5 g/kg. Each group of 10 animals, with 18.3±1.5 grams of weight, half male and half female, were orally administrated with the prepared 0.5% CMC liquid. The results showed that the two groups had normal activity and no obvious toxicity. There was no significant difference in toxicity between the two groups.

Oxiracetam is commercially available. Its properties on pharmakinetics and toxicology are well-known.

C. Experimental Study of R-Oxiracetam Against Epilepsy

The rat epilepsy models were treated with lithium chloride-pilocarpine and used levetiracetam as positive control. The duration of acute seizures of lithium chloride-pilocarpine epilepsy model rat was selected as the observation time, and prophylactic administration was used.

1 Experimental Material and Systems

1.1 Experimental Materials

Testing Sample:

R-oxiracetam, raw material, milky white powder, lot number 20100205, soluble in water. levorotatory oxiracetam, white powder, lot number 091202, soluble in water.

The above were purchased from Chongqing Dongze Pharmaceutical Science and Technology Co., Ltd.

Positive Control:

Levetiracetam tablet is used in plus treatment for adults and children over 4 years of age epilepsy patients with partial seizure, having traits for blue oval film coated tablets, 0.25 g/tablet, and manufactured by UCB Pharma S.A. The initial dose of an adult is 500 mg/times, twice daily, that is, 1000 mg/60 kg, 16.7 mg/kg. The dose of a rat is converted to 100 mg/kg according to body surface area.

S-oxiracetam and R-oxiracetam were prepared with pure water before use. The levetiracetam tablet was prepared with 0.5% CMC at the desired concentration before use. 0.5% CMC is used as solvent reference substance, lot number 2014010801, manufactured by Chengdu Kelong Chemical Reagent Factory.

Reagents:

Nitrochrome pilocarpine (pilocarpine) eye drops, 5 ml: 25 mg, lot number: 14101901, Shandong Dr. Lun Fu Ruida Pharmaceutical Co., Ltd, diluted to 3 mg/ml of concentration with NS before use.

Anhydrous lithium chloride, white particles, molecular weight 42.39, Chengdu Kelong Chemical Reagent Factory, prepared with NS to 12.7 mg/ml as spare.

1.2 Laboratory System

Laboratory animals

Species/strains: SD rats

Level: SPF level

Supply unit: Chengdu Dasuo Biological Technology Co., Ltd

Production license number: SOCK (Sichuan) 2008-24

Purchase of animal sex and quantity: 50, half male and half female;

Weight range when purchased: 120˜150 g

Animal feeding and management

Environmental conditions for animal feeding and management
Feeding room: barrier system animal laboratory
Environmental class: barrier system

Temperature: 20˜25° C.

Relative humidity: 40˜70%;

Number of ventilation: 10-15 times/hour

Lighting time: 12 h/12 h alternating lighting every day.

Animal cages: PC polycarbonate cartridges (L×W×H: 460 mm×315 mm×210 mm), by the Suzhou City, Suzhou and Hangzhou animal plant production.

Litter: the corncob as litter, provided by the Suzhou Shuang Lion Experimental Animal Feed Technology Co., Ltd., heated by high temperature and high pressure steam sterilization for animal use.

Cage updates frequency: 2-3 times a week to replace.

Cleaning and disinfection: cleaning was carried out after the end of daily test operation and feeding work, and then disinfection by the animal management room. Disinfectants were alternately selected 84 disinfectants, Lysol and bromogeramine, once a week to exchange the type of disinfectants.

Quarantine

The quarantine period is set to 7 days. During the quarantine period, the daily appearance and the general state of the animals are observed and recorded. If any animals acted abnormal, the test may be affected. The animal should be removed from the test and shall not be used.

Fodder

Rat full price pellet feed provided by the Institute of Laboratory Animals of Sichuan Academy of Medical Science & Sichuan Provincial Research's Hospital, which were meet GB14924.3-2001 standard were used after radiation of Co60.

Feeding method: free intake

Drinking water

High pressure steam sterilization tap water was freely taken by animal drinking water bottles.

2 Experiment Methods

2.1 Animal Identification and Numbering

The tail marking method was used during the quarantine period, that is, the animal mark is indicated by the mark stroke on the tail. Prior to administration, the animals were grouped by body weight stratification randomized grouping and labeled with picric acid staining.

2.2 Dose Setting

Animals were randomly divided into 4 groups, 10/group. Levetiracetam was selected as a positive control, 1.5 times of clinical dose were chosen, that is, 150 mg/kg. The clinical dose of an adult is 800 mg/times, twice daily, that is, 1600 mg/60 kg, 26.7 mg/kg. The dose of a rat is converted to 160 mg/kg according to body surface area. R-oxiracetam and levorotatory oxiracetam were selected half of the dose, that is, 80 mg/kg, and 1.5 times is 120 mg/kg. The dose was 1 ml/100 g, and the rats were treated with continuous administration for 7 days. As levetiracetam administration of 1.3 h (80 min), the plasma concentration peak, so we choose the last 80 minutes after the last administration of modeling. As administration of levetiracetam for 1.3 h (80 min), the plasma concentration peak was reached, so each group was selected after the last 80 minutes after the start of modeling.

The specific dose settings are as follows:

| | Serial number | dose | Concentration | Clinical dose |
|---|---|---|---|---|
| Groups | of each dog | (mg/kg) | (mg/ml) | multiple |
| Model Control | 1F01~1F05, | — | 0.5% CMC | — |
| Group | 1M01~1M05 | | | |
| Levetiracetam | 2F01~2F05, | 150 | 15 | 1.5 |
| Group | 2M01~2M05 | | | |
| S-Oxiracetam | 3F01~3F05, | 120 | 12 | 1.5 |
| Group | 3M01~3M05 | | | |
| R-Oxiracetam | 4F01~4F05, | 120 | 12 | 1.5 |
| Group | 4M01~4M05 | | | |

2.3 Test Methods

Modeling method: After several previous test, the following modeling method was chosen: Animal ip. Lithium chloride 3 mmol/kg (1.5 ml/L) 127 mg/kg (12.7 mg/ml, 1 ml/100 g body weight), 24 h, 37.5 mg/kg pilocarpine three times ip. mg/kg (2.5 mg/ml, 0.5 ml/100 g body weight) at intervals of 10 minutes.

As the number of animals per observation was limited, the test was divided into four batches.

2.4 Observe Indicators

The seizure status, seizure level, latency, duration of seizures, seizures and mortality were recorded within 3 hours after modeling, and compared with the model control group,

3 Test Results

All rats had symptoms of cholinergic peripheral nerve stimulation, that is, vertical hair, salivation, blood tears, after 5 to 10 min of the first injection of pilocarpine. Also, varying degrees of the following performance appeared, including: less moving, stare, mouth automatic disease, nod, and blink, wet dog-like shaking.

3.1 Mortality Rate and Number of SE Animals

| | | | | Mor- | | SE |
|---|---|---|---|---|---|---|
| | Numbers | | Number | tality | Number | occur- |
| | of | Dose | of | rate | of SE | rence |
| Groups | Animals | (mg/kg) | Deaths | (%) | Animals | rate (%) |
| Model Control | 10 | — | 4 | 40 | 6 | 60 |
| Group | | | | | | |
| Levetiracetam | 10 | 150 | 5 | 50 | 7 | 70 |
| group | | | | | | |
| S-Oxiracetam | 10 | 120 | 3 | 30 | 4 | 40 |
| Group | | | | | | |
| R-Oxiracetam | 10 | 120 | 1 | 10 | 2 | 20 |
| Group | | | | | | |

Effect of R-oxiracetam on mortality rate and SE occurrence rate to the lithium chloride-pilocarpine-made epilepsy model rats is shown as FIG. 3.

The results of the above test showed that the animals in each group appeared varying degrees of alkali-related peripheral nerve stimulation symptoms and different levels of convulsions. There was a certain mortality rate within 1 to 7 days after modeling. The mortality was 40% for the model control group, 50% for the levetiracetam group, 30% for the S-oxiracetam group and 10% for the R-oxiracetam group. The percentages of status epilepticus in each group were 60%, 70%, 40%, 20%. The percentage of SE in R-oxiracetam animals was significantly lower than in the control group. It is indicated that R-oxiracetam could reduce the percentage of epilepsy in pilocarpine epilepsy model rats and the mortality rate, and had protective effect on epilepsy rats.

3.2 Different Stages of Animals

| | | | Occurrence rate (%) | | | | |
|---|---|---|---|---|---|---|---|
| | Number of | Dose | Stage | Stage | Stage | Stage | Stage |
| Groups | animals | (mg/kg) | I | II | III | IV | V |
| Model Control | 10 | — | 10 | 10 | 9 | 8 | 6 |
| Group | | | | | | | |
| Levetiracetam | 10 | 150 | 10 | 9 | 8 | 7 | 7 |
| group | | | | | | | |
| S-Oxiracetam | 10 | 120 | 10 | 10 | 5 | 4 | 4 |
| Group | | | | | | | |
| R-Oxiracetam | 10 | 120 | 10 | 10 | 5 | 2* | 2 |
| Group | | | | | | | |

Note:
*indicates that the model control group by X² test, P < 0.05

The effect of R-oxiracetam on occurrences of different stages is shown in FIG. 4.

In the above tests, all the animals in the group had all the symptoms of stage I and II, half of the S-oxiracetam group and R-oxiracetam group had no symptoms of stage III and above. The number of animals having stage IV in the R-oxiracetam group was 20%, significantly different from the model control group (P<0.05). It is indicated that R-oxiracetam group can significantly reduce the number of animals having symptoms of stage IV in the model rats and inhibit seizures.

3.3 Latency of Different Stages

| | | | Latency (min) | | | | |
|---|---|---|---|---|---|---|---|
| Groups | Number of animals | Dose (mg/kg) | Stage I | Stage II | Stage III | Stage IV | Stage V |
| Model Control | 10 | — | 14.3 ± 5.3 | 22.5 ± 5.3 | 35.9 ± 7.0 | 39.6 ± 9.5 | 73.5 ± 24.2 |
| Group | | | | | | | |
| Levetiracetam | 10 | 150 | 17.6 ± 4.1 | 22.6 ± 5.8 | 34.6 ± 9.4 | 33.6 ± 8.3 | 74.3 ± 24.3 |
| group | | | | | | | |
| S-Oxiracetam | 10 | 120 | 16.9 ± 5.4 | 26.7 ± 6.5 | 35.4 ± 5.8 | 37.8 ± 7.4 | 64.5 ± 23.2 |
| Group | | | | | | | |
| R-Oxiracetam | 10 | 120 | 19.6 ± 4.4 | 34.7 ± 15.2 | 40.2 ± 12.1 | 35.0 ± 2.8 | 38.0 ± 2.8 |
| Group | | | | | | | |

Experimental data showed that the animals in the R-oxiracetam group appear the trend for longer latency of stage I, II and III symptoms than the model control group. It is indicated that R-oxiracetam can prolong the time of partial epileptic seizures of the model animals, and has protective effects on epilepsy model rats.

3.4 Number of Seizures and Duration at Different Stages

| | | | Number of seizures (times) | | | duration (min) | |
|---|---|---|---|---|---|---|---|
| Group | Numbers of Animals | Dose (mg/kg) | Stage I | Stage II | Stage III | Stage IV | Stage V |
| Model Control | 10 | — | 2.0 ± 1.6 | 6.9 ± 5.3 | 4.7 ± 2.2 | 16.5 ± 15.2 | 72.0 ± 14.4:(*) |
| Group | | | | | | | |
| Levetiracetam | 10 | 150 | 1.8 ± 0.8 | 4.4 ± 3.7 | 3.6 ± 2.3 | 23.3 ± 6.6 | 63.3 ± 19.0 |
| group | | | | | | | |
| S-Oxiracetam | 10 | 120 | 2.2 ± 1.0 | 5.2 ± 3.1 | 5.6 ± 4.2 | 27.5 ± 12.1 | 79.8 ± 29.5 |
| Group | | | | | | | |
| R-Oxiracetam | 10 | 120 | 2.3 ± 1.2 | 6.8 ± 3.7 | 6.4 ± 3.6 | 10.0 ± 1.4 | 26.5 ± 0.7(*) |
| Group | | | | | | | |

Note:
(*)indicates that the model control group by analysis of variance, P < 0.05

The duration of stage V seizures of animals in the R-oxiracetam group was significantly lower than that in the control group (P<0.05). It is indicated that R-oxiracetam group can significantly reduce the timer of stage IV symptoms occurred on the model rats, and had protective effects on epilepsy model rats inhibit seizures.

Pentetrazol (PTZ) was used to ignite the rat epilepsy model with Sodium valproate as a positive control, in order to observe whether R-oxiracetam has the antiepileptic effect.

### 1. Testing Sample, Reference Substances and Reagents

Testing Sample:

R-oxiracetam, raw materials, white powder, lot number 20150603, content:99.92%, soluble in water. provide by Chengdu Biotoppharma Medical Technology Co., Ltd.

Positive Control:

Sodium valproate, lot number 140440, specified as 0.5 g/tablet, Sanofi Hangzhou Pharmaceutical Co., Ltd. Gastrointestinal absorption of oral administration was quick and complete. The peak plasma concentration was reached after the administration about 1 to 4 hours, with bioavailability of nearly 100%. The daily maximum dose for human is 30 mg/kg, and for a rat dose is 185 mg/kg by conversion of body surface area.

R-oxiracetam was prepared by pure water before use. Sodium valproate tablets ewas formulated by 0.5% CMC into the desired concentration of the suspension solution before use. 0.5% CMC is used as reference substance of vehicles, lot number 2014010801, Chengdu Kelong Chemical Reagent Factory.

Reagents:

Pentetrazol (PTZ), Sigma, MKBT2860V, white powder, prepared with NS into a concentration of 7 mg/ml and reserved before use

### 2. Laboratory System

### 2.1 Laboratory Animals

Species/strains: SD rats

Level: SPF level

Supply unit: Chengdu Dasuo Biological Technology Co., Ltd

Production license number: SOCK (Sichuan) 2008-24

Purchase of animal sex and quantity: 40, male;

Weight range when purchased: 120˜150 g

### 2.2 Animal Feeding and Management

### 2.2.1 Environmental Conditions for Animal Feeding and Management

Feeding room: barrier system animal laboratory

Environmental class: barrier system

Temperature: 20˜25 ° C.

Relative humidity: 40˜70%;

Number of ventilation: 10-15 times/hour

Lighting time: 12 h/12 h alternating lighting every day.

Animal cages: PC polycarbonate cartridges (L×W×H: 460 mm×315 mm×210 mm), by the Suzhou City, Suzhou and Hangzhou animal plant production.

Litter: the corncob as litter, provided by the Suzhou Shuang Lion Experimental Animal Feed Technology Co., Ltd., heated by high temperature and high pressure steam sterilization for animal use.

Cage updates frequency: 2-3 times a week to replace.

Cleaning and disinfection: cleaning was carried out after the end of daily test operation and feeding work, and then disinfection by the animal management room. Disinfectants were alternately selected 84 disinfectants, Lysol and bromogeramine, once a week to exchange the type of disinfectants.

### 2.2.2 Quarantine

The quarantine period is set to 7 days. During the quarantine period, the daily appearance and the general state of the animals are observed and recorded. If any animals acted abnormal, the test may be affected. The animal should be removed from the test and shall not be used.

### 2.2.3 Fodder

Rat full price pellet feed provided by the Institute of Laboratory Animals of Sichuan Academy of Medical Science & Sichuan Provincial Research's Hospital, which were meet GB14924.3-2001 standard were used after radiation of Co60.

Feeding method: free intake

### 2.2.4 Drinking Water

High pressure steam sterilization tap water was freely taken by animal drinking water bottles.

### 3 Experiment Methods

### 3.1 Animal Identification and Numbering

The tail marking method was used during the quarantine period, that is, the animal mark is indicated by the mark stroke on the tail. Prior to administration, the animals were grouped by body weight stratification randomized grouping and labeled with picric acid staining.

### 3.2 Modeling Method

Rats were injected intraperitoneally with PTZ at a dose of 35 mg/kg (7 mg/ml, 0.5 ml/100 g body weight) for sub-convulsions three times weekly. The animals are weighed before each ip, and observed for behavioral changes after ip. within 1-2 h. Behavioral scoring was carried out according to the revised Racine score. Three consecutive occurrences of seizures of stage IV and above indicate successful ignition.

According to the previous tests, generally in the tenth time of the PTZ injection, three seizures of stage IV occur consecutively, means successful modeling.

### 3.3 Dose Setting

The animals successfully ignited were chosen to be grouped. The animals were randomly divided into 4 groups, 8-10/group. Sodium valproate was selected as a positive control. 3 times of clinical dose were chosen, that is, 500 mg/kg. According to the results of the previous tests, 120 mg/kg was selected as the low dose of R-oxiracetam, and 3 times thereof, 360 mg/kg as the high dose. The dosage is 1 ml/100 g, continuous intragastrical administration of 10 days. Specific dose settings are as follows:

TABLE 1
Dose Settings

| | Dose | Concentration | Clinical dose |
|---|---|---|---|
| Groups | (mg/kg) | (mg/ml) | multiple |
| Model Control | — | 0.5% CMC | — |
| Group | | | |
| Sodium valproate | 500 | 50 | 3 |
| Control Group | | | |
| Low-dose | 120 | 12 | — |
| R-Oxiracetam | | | |
| Group | | | |
| High-dose | 360 | 36 | — |
| R-Oxiracetam | | | |
| Group | | | |

### 3.4 Test Methods

One hour after administration of each group of the animals (according to the peak time of sodium valproate) ip. PTZ 35 mg/kg. The animals are observed for behavioral changes within 2 h. Behavioral scoring was carried out according to the revised Racine score, continuous administration for 10 days, grading standards as mentioned above.

### 3.5 Observe Indicators

The seizures were recorded daily ip PTZ within 2 h. The seizure levels, latencies, mortality were recorded, and compared with the model control group.

### 4 Test Results

### 4.1 Model Selection

A total of 41 animals died during the animal modeling. Twenty-seven animals that were successfully ignited were screened and grouped according to the seizure stages, with about 7 animals in each group.

### 4.2 Mortality Rate

During the administration, two animals died in the model group, with a mortality rate of 25%. No animal died in the low-dose group of R-oxiracetam and in the high-dose group and the positive control group. The chi-square test, no statistical difference.

TABLE 2
Impact on Mortality

| | Numbers of | | Numbers of | mortality |
|---|---|---|---|---|
| | animals | Dose | deaths | rate |
| Groups | (Number) | (mg/kg) | (Number) | (%) |
| Model Control | 8 | — | 2 | 25 |
| Group | | | | |
| Sodium | 6 | 500 | 0 | 0 |
| valproate control | | | | |
| group | | | | |
| Low-dose | 7 | 120 | 1 | 14.3 |
| R-Oxiracetam | | | | |
| Group | | | | |
| High-dose | 6 | 360 | 0 | 0 |
| R-Oxiracetam | | | | |
| Group | | | | |

### 4.3 Occurrence Rates of Stage IV or Above Seizures

TABLE 3-1
occurrence rates of stage IV or above seizures (%)

| Groups | Dose (mg/kg) | D1 | D2 | D3 | D4 | D5 |
|---|---|---|---|---|---|---|
| Model control | — | 7/8 (87.5) | 6/7(85.7) | 5/7 (71.4) | 5/6 (83.3) | 5/6 (83.3) |
| group | | | | | | |
| Sodium valproate | 500 | 4/6 (66.7) | 3/6(50.0) | 3/6 (50.0) | 2/6 (33.3) | 2/6 (33.3) |
| control group | | | | | | |
| Low-dose | 120 | 4/7 (57.1) | 4/6(66.7) | 3/6 (50.0) | 2/6 (33.3) | 2/6 (33.3) |
| R-Oxiracetam | | | | | | |
| Group | | | | | | |
| High-dose | 360 | 4/6 (66.7) | 3/6(50.0) | 3/6 (50.0) | 2/6 (33.3) | 1/6 (16.7) |
| R-Oxiracetam | | | | | | |
| Group | | | | | | |

TABLE 3-2
Occurrence rates of animal with Stage IV or above seizures (%)

| Groups | Dose (mg/kg) | D6 | D7 | D8 | D9 | D10 |
|---|---|---|---|---|---|---|
| Model control | — | 5/6 (83.3) | 5/6 (83.3) | 5/6 (83.3) | 4/6 (66.7) | 4/6 (66.7) |
| group | | | | | | |
| Sodium | 500 | 2/6 (33.3) | 1/6* (16.7) | 0/6** (0) | 1/6 (16.7) | 0/6* (0) |
| valproate | | | | | | |
| control group | | | | | | |
| Low-dose | 120 | 2/6 (33.3) | 1/6* (16.7) | 1/6* (16.7) | 0/6* (0) | 0/6* (0) |
| R-Oxiracetam | | | | | | |
| Group | | | | | | |
| High-dose | 360 | 1/6 (16.7) | 0/6** (0) | 0/6** (0) | 0/6* (0) | 0/6* (0) |
| R-Oxiracetam | | | | | | |
| Group | | | | | | |

Note:
Compared with the model control group, by chi-square test,
*indicates P < 0.05;
**indicates P < 0.01.

The results are shown in Tables 3-1, 3-2 and 5. The results showed that during the administration of the model group animals, the frequency of symptoms of stage IV or above remained at a high level (80%). Compared with the model group, there was no significant difference among the three groups in the early administration. From D7 (D is the number of days, D7 is the seventh day), the occurrence rate was significantly reduced until the end of administration, with statistical differences. The high-dose R-Oxiracetam group of animals from D7 have only stage II-III seizures, and no stage IV, V seizures occurs. From the occurrence rate in the diagram can also be seen that the seizures of stage IV and V of the three administration group was significantly lower than the model control group, the high-dose R-Oxiracetam group has the best effect.

### 4.4 Latency

The results are shown in Table 4-1 and 4-2. In the early administration, each administration group compared with the model group, the onset of latency was no significant difference. Starting from D4, the latency of animals in the positive group and the high-dose R-Oxiracetam group was significantly prolonged (P<0.05, P<0.01), and continued until the end of administration. Low-dose R-Oxiracetam group starting from D6, the latency was significantly longer than the model control group (P<0.05, P<0.01), until the end of administration.

TABLE 4-1
Seizure latency data (m, X ± SD)

| Groups | Dose (mg/kg) | D1 | D2 | D3 | D4 | D5 |
|---|---|---|---|---|---|---|
| Model Control | — | 3.88 ± 1.25 | 4.25 ± 0.71 | 4.25 ± 1.04 | 4.00 ± 0.76 | 4.38 ± 0.92 |
| Group | | | | | | |
| Sodium valproate | 500 | 4.00 ± 1.41 | 5.67 ± 2.25 | 5.67 ± 1.37 | 6.33 ± 1.63* | 6.83 ± 1.47** |
| Control Group | | | | | | |
| Low-dose | 120 | 3.57 ± 1.51 | 4.57 ± 1.72 | 5.00 ± 1.15 | 4.86 ± 1.07 | 5.43 ± 0.98 |
| R-Oxiracetam | | | | | | |
| Group | | | | | | |
| High-dose | 360 | 3.83 ± 1.17 | 5.00 ± 0.89 | 6.17 ± 1.17 | 6.50 ± 1.05* | 7.17 ± 0.75* |
| R-Oxiracetam | | | | | | |
| Group | | | | | | |

TABLE 4-2
Seizure latency data (m, X ± SD)

| Groups | Dose (mg/kg) | D6 | D7 | D8 | D9 | D10 |
|---|---|---|---|---|---|---|
| Model Control | — | 4.75 ± 1.04 | 4.63 ± 0.92 | 4.88 ± 0.83 | 4.38 ± 0.74 | 4.75 ± 0.71 |
| Group | | | | | | |
| Sodium valproate | 500 | 7.33 ± 1.03** | 7.50 ± 1.05** | 7.83 ± 0.75** | 7.67 ± 0.82** | 8.33 ± 0.82** |
| Control Group | | | | | | |
| Low-dose | 120 | 6.29 ± 1.11 * | 6.86 ± 1.07** | 7.14 ± 1.07** | 7.71 ± 0.76** | 8.43 ± 0.98** |
| R-Oxiracetam | | | | | | |
| Group | | | | | | |
| High-dose | 360 | 7.83 ± 0.75** | 8.33 ± 1.03** | 8.83 ± 0.98** | 8.83 ± 0.75** | 9.83 ± 0.75** |
| R-Oxiracetam | | | | | | |
| Group | | | | | | |

Note:
Compared with the model control group, one-way analysis of variance,
* means P < 0.05;
**means P < 0.01.

The results showed that after modeling, there were different degrees of alkali peripheral nerve stimulation symptoms and different levels of seizures in each group of animals, and there was a certain mortality ( 3/41) during the modeling. Animals were also killed during the dosing period, with a mortality rate of 25% in the model control group, 0 in the positive control group, 14.3% in the low-dose R-Oxiracetam group, 0 in the high-dose R-Oxiracetam group, Sodium soda group mortality rate of 0. In absolute values, it has a protective effect on seizures in animals.

On the stage IV or above seizures and the latency of the results, the R-Oxiracetam and positive drugs on the epilepsy model have significant protective effects, the performance in the middle and late administration of the stage IV seizures were significantly lowered, the latency was significantly longer, of which the role of the high dose R-Oxiracetam was most obvious.

The test results showed that the R-Oxiracetam has a curative effect on the epilepsy model, the performance in the mortality rate and the stage IV or above seizures were lower than the model control group, and the latency was significantly longer than the model control.

The invention has the advantages that:

The inventor accidentally discovered that R-oxiracetam has an antiepileptic activity and had a significant inhibitory effect on acute epilepsy, especially has a significant inhibitory effect on acute epilepsy seizures. R-oxiracetam has a good inhibitory effect on generalized epilepsy seizure, partial epilepsy seizure and status epilepticus, and R-oxiracetam has high bioavailability and low toxicity, as well as suitable for further development as an antiepileptic drug. The invention uses the R-oxiracetam having more than 99.0% of purity, effectively excluding the interference from other components. R-oxiracetam is a single active ingredient, making the quality of the drug easier to control, while the effect is clearer. The composition of the R-oxiracetam provided by the invention has strong adaptability and can be simply prepared, thereby being advantageous for industrialization.

### BRIEF DESCRIPTION OF THE DRAWINGS

FIG. 1 is a diagram of standard curves of oxiracetam.

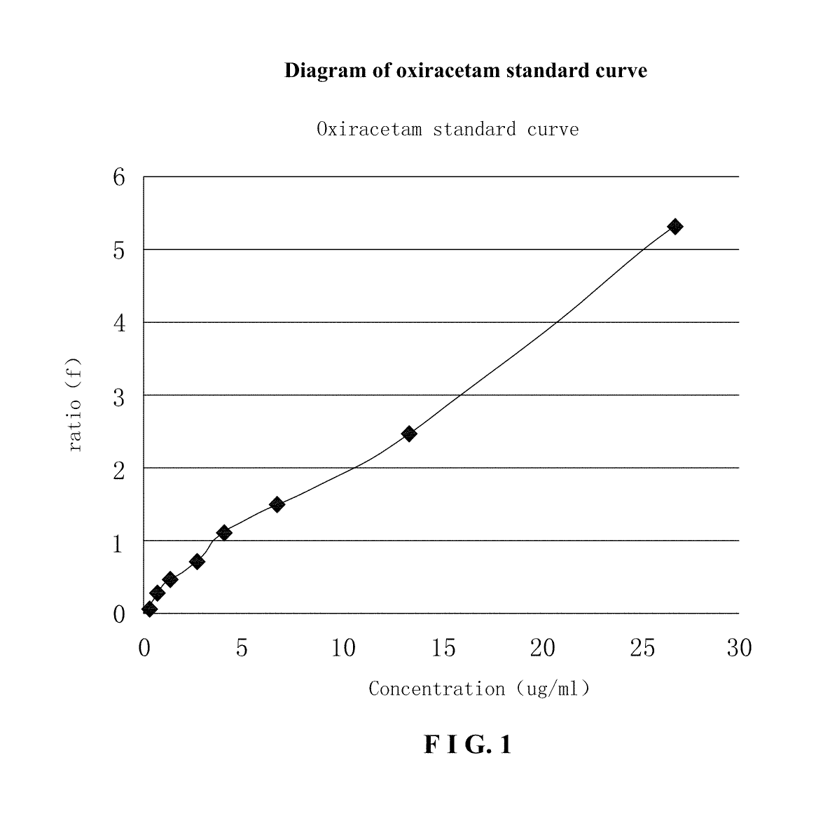

FIG. 2 is a diagram of standard curves of R-oxiracetam.

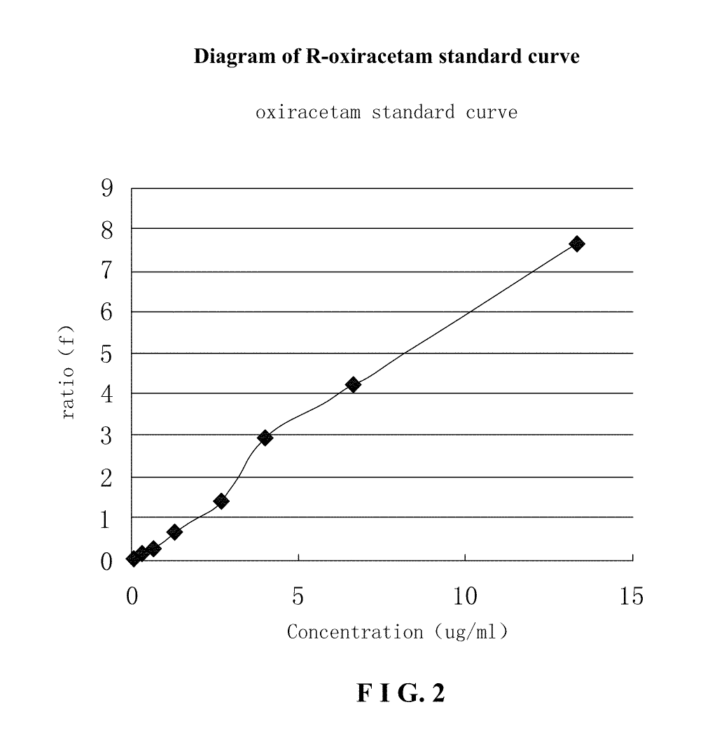

FIG. 3 is a bar chart of effects of R-oxiracetam on mortality rate and SE occurrence rate of the lithium chloride-pilocarpine epilepsy model rats.

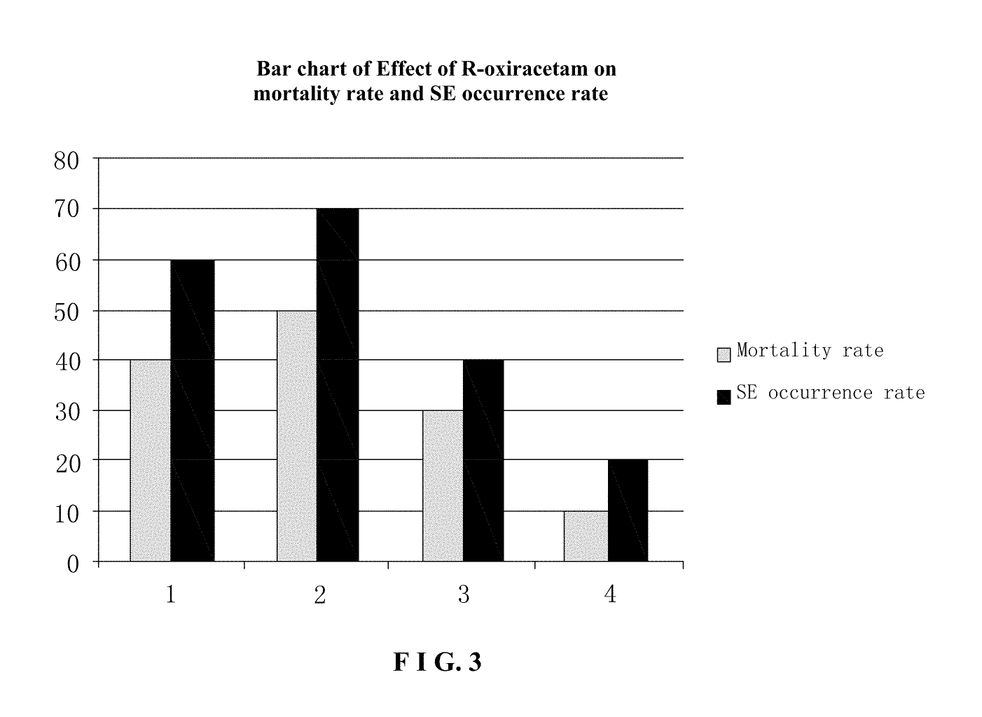

FIG. 4 a diagram of effects of the lithium chloride-pilocarpine epilepsy model rats on occurrences of different stages

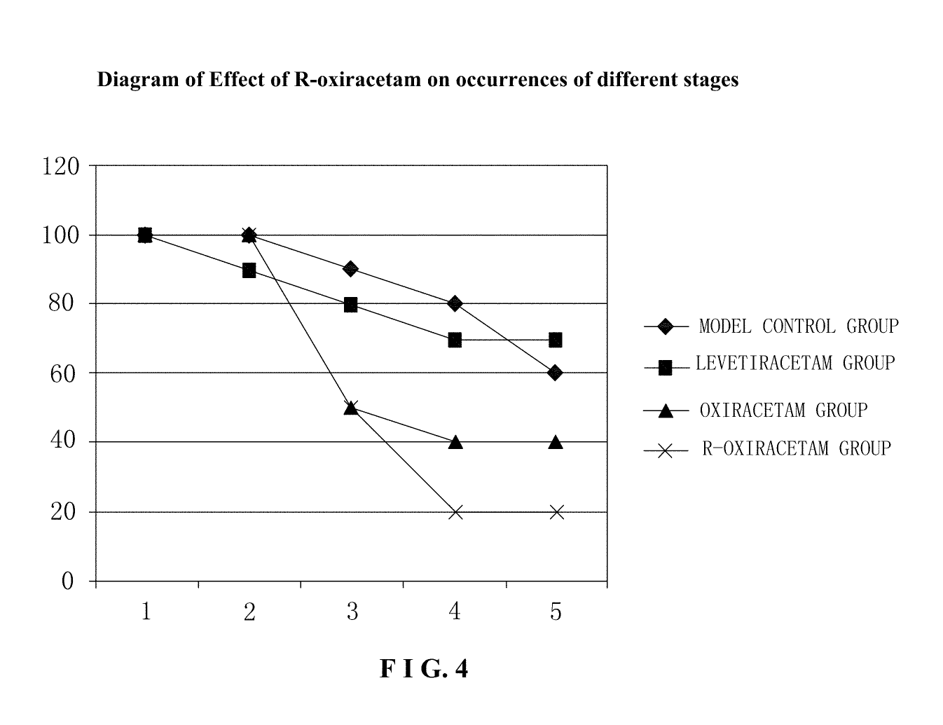

FIG. 5 a diagram of occurrence rate of stage IV or above seizures of pentetrazol (PTZ)-ignited rat epilepsy model with R-oxiracetam.

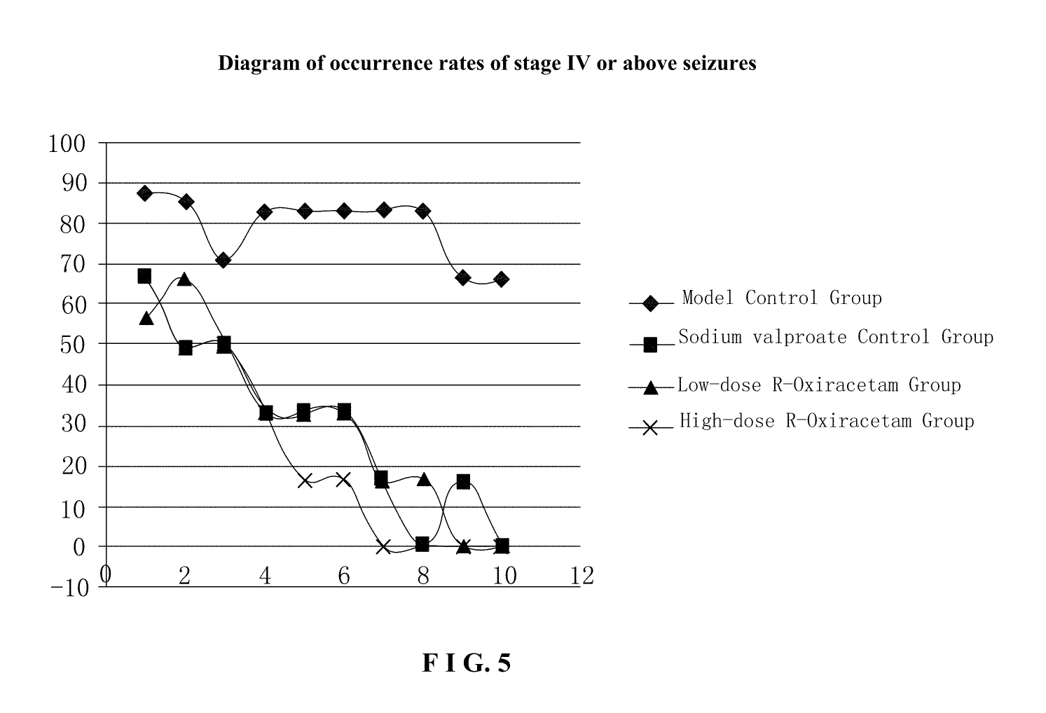

### DETAILED DESCRIPTION OF THE EMBODIMENTS

Hereinafter, several embodiments of the present invention will be described, but the present invention is not limited thereto.

### EXAMPLE 1

Raw Material Composition:

| | | |
|---|---|---|
| (a) R-oxiracetam (99.5% of purity) | 200 | mg/tablet |
| (b) Lactose | 80 | mg/tablet |
| (c) Microcrystalline cellulose | 70 | mg/tablet |

For example, to produce 1000R-oxiracetam capsules, the specific preparation method is: Raw materials were passed over 80 mesh sieve. The prescriptive amount of R-oxiracetam, lactose, microcrystalline cellulose, are taken and mixed to directly fill into capsules.

### EXAMPLE 2

Raw Material Composition:

| | | |
|---|---|---|
| (a) R-oxiracetam (99.6% of purity) | 200 | mg/tablet |
| (b) Starch | 34 | mg/tablet |
| (c) Microcrystalline cellulose | 60 | mg/tablet |
| (d) Talcum powder | 6 | mg/tablet |

| | |
|---|---|
| (e) 2% hydroxypropyl methyl cellulose (K4M model) | adequate amount |

For example, to produce 1000 R-oxiracetam tablets, the specific preparation method is: Raw materials were passed over 80 mesh sieve. The prescriptive amount of R-oxiracetam, starch, and microcrystalline cellulose were taken and mixed homogenously, then added to soft material made by 2% HPMC solution. After granulating, drying, granulating, the granules were added prescriptive amount of talcum powder and then mixing and tableting.

### EXAMPLE 3

Raw Material Composition:

| | | |
|---|---|---|
| (a) R-oxiracetam (99.0% of purity) | 200 | mg/tablet |
| (b) Lactose | 80.8 | mg/tablet |
| (c) Sodium carboxymethyl starch | 72 | mg/tablet |
| (d) Talcum powder | 7.2 | mg/tablet |

| | |
|---|---|
| (e) 10% polyvinylpyrrolidone | adequate amount |

For example, to produce 1000 R-oxiracetam capsules, the specific preparation method is: Raw materials were passed over 80 mesh sieves. The prescriptive amount of R-oxiracetam, lactose, sodium carboxymethyl starch were taken and mixed homogenously, then added to soft material made by 10% PVP ethanol solution. After granulating, drying, granulating, the granules were added prescriptive amount of talcum powder and then mixing and filling into capsules.

### EXAMPLE 4

For example, to produce R-oxiracetam injection solution, the specific preparation method is: 50 g of R-oxiracetam (99.3% of purity), 150 g of glucose, 500 ml of water for Injection are dissolved in a dilution can, and then mixed under 50˜60° C. until totally dissolved. The dissolved solution was cooled to 25° C. Charcoal was added into the dissolved solution for decolorization. The charcoal was filtered and removed, and then phosphate buffer was added into the dissolved solution to adjust its pH value to 4.0. Then, the water for injection was added to make 5000 ml of the diluted solution. The diluted solution was sealed and sterilized under 105° C. for 30 minutes to obtain the R-oxiracetam injection solution.

1. A method of preventing or treating epilepsy, comprising administering to a subject in need a drug comprising a therapeutically effective amount of R-oxiracetam compound, and pharmaceutically acceptable excipients.

2. A method of preventing or treating acute epilepsy seizure, comprising administering to a subject in need a drug comprising a therapeutically effective amount of R-oxiracetam compound, and pharmaceutically acceptable excipients.

3. A method of preventing or treating generalized epilepsy seizure, comprising administering to a subject in need a drug comprising a therapeutically effective amount of R-oxiracetam compound, and pharmaceutically acceptable excipients.

4. A method of preventing or treating partial epilepsy seizure, comprising administering to a subject in need a drug comprising a therapeutically effective amount of R-oxiracetam compound, and pharmaceutically acceptable excipients.

5. A method of preventing or treating status epilepticus, comprising administering to a subject in need a drug comprising a therapeutically effective amount of R-oxiracetam compound, and pharmaceutically acceptable excipients.

6. The method as claimed in claim 1, characterized in that the R-oxiracetam compound or a composition containing the R-oxiracetam compound is administered at a dose of not less than 400 mg/day.

7. The method as claimed in claim 2, characterized in that the R-oxiracetam compound or a composition containing the R-oxiracetam compound is administered at a dose of not less than 400 mg/day.

8. The method as claimed in claim 3, characterized in that the R-oxiracetam compound or a composition containing the R-oxiracetam compound is administered at a dose of not less than 400 mg/day.

9. The method as claimed in claim 4, characterized in that the R-oxiracetam compound or a composition containing the R-oxiracetam compound is administered at a dose of not less than 400 mg/day.

10. The method as claimed in claim 5, characterized in that the R-oxiracetam compound or a composition containing the R-oxiracetam compound is administered at a dose of not less than 400 mg/day.

11. The drug as claimed in any one of claim 1, characterized in that the optical purity of the R-oxiracetam compound is 99.0% or more, based on weight percent.

12. The method as claimed in claim 2, characterized in that the optical purity of the R-oxiracetam compound is 99.0% or more, based on weight percent.

13. The method as claimed in claim 3, characterized in that the optical purity of the R-oxiracetam compound is 99.0% or more, based on weight percent.

14. The method as claimed in claim 4, characterized in that the optical purity of the R-oxiracetam compound is 99.0% or more, based on weight percent.

15. The method as claimed in claim 5, characterized in that the optical purity of the R-oxiracetam compound is 99.0% or more, based on weight percent.
